(19) United States
(12) Patent Application Publication
(10) Pub. No.: US 2012/0029425 A1
(43) Pub. Date: Feb. 2, 2012

(54) COMBINED CATHETER TIP AND INFLATION BALLOON

(76) Inventors: Vincent G. Copa, Minnetonka, MN (US); Robert Rykus, JR., Edina, MN (US)

(21) Appl. No.: 13/132,215

(22) PCT Filed: Dec. 1, 2009
(86) PCT No.: PCT/US09/66235

§ 371 Date: Aug. 16, 2011

(30) Foreign Application Priority Data
Dec. 1, 2008 (US) 61118788

(51) Int. Cl. A61M 29/00; B29C 45/14

(52) U.S. Cl. 604/101.01; 264/250

(57) ABSTRACT

An integrated catheter tip that is fabricated to include an inflation balloon that is formed/bonded to the catheter tip as part of an initial molding operation. A tip portion of the integrated catheter tip is formed of a first high durometer polymer while the inflation balloon is formed using a second low durometer polymer. Utilizing a two-step molding operation, the first high durometer polymer can be injected to form the catheter tip and subsequently allowed to cool. Next, the second low durometer polymer is injected to form the inflation balloon and to bond the inflation balloon to the catheter tip so as to create an integrated catheter tip. Once the integrated catheter tip has been formed, the integrated catheter tip can be attached to a catheter body utilizing a conventional attachment method.

### PRIORITY CLAIM

The present application claims priority to U.S. Provisional Application Ser. No. 61/118,788, filed Dec. 1, 2008, and entitled “COMBINED BALLOON TIP”, which is herein incorporated by reference in its entirety.

### FIELD OF THE DISCLOSURE

The invention relates generally to a catheter including an inflation balloon proximate a catheter tip. More specifically, the present invention relates to a catheter tip and inflation balloon that can be combined through a molding process to create a combined tip assembly for attachment to a catheter body.

### BACKGROUND OF THE DISCLOSURE

A medical catheter is essentially an artificial tube or lumen that is inserted into a body lumen to provide access to an area to be examined or treated. Typically, catheters comprise a treatment or manipulation apparatus at a proximal end that allows a medical professional to advance the catheter to the appropriate area and to examiner or provide medical treatment to tissue proximate the body lumen at that location. While a treatment end of the catheter is adapted to provide medical treatment within the body lumen, the treatment end can suffer from an inability to be easily navigated to the targeted tissue unassisted because of its diameter, rigidness or shape. In these instances, a tip can be attached to treatment end. Generally, the tip is smaller in diameter, softer or more flexible than the operational end so as to ease the movement of the catheter through the body cavity and minimize damage to the surrounding tissue as the catheter is navigated to the targeted tissue.

Depending upon the style of the catheter, attachment of the tip can be accomplished in different ways. The tip is typically affixed to the end of the catheter body by aligning the end of the tip with the end of the catheter body and bonding the ends of the tip and catheter body together with a bonding agent such as adhesive or plasticizer. Open ended catheters have an exposed catheter lumen that may be sufficiently large in diameter to allow at least a portion of the tip to be inserted into the catheter body. In contrast, closed ended or “blind ended” catheters have closed catheter lumen and cannot receive a portion of the tip into the catheter body. As a result, the tip and the blind ended catheter body must be aligned manually and retained in the aligned position until bonded together.

When the medical catheter is being used to dispense treatment to the targeted tissue, the distal treatment end can be fabricated to include a wide variety of delivery mechanisms. For example, a distal treatment end of a catheter body can include injection ports for delivering a therapeutic agent to the tissue, fiber optic ports for delivering laser energy to the tissue, temperature probes for delivering either heating or cooling at the tissue or even microwave antennas to provide targeted microwave energy to the tissue rather than transmitting the microwave energy through the patient's skin to attempt or reach the tissue. In addition to delivering therapy to the targeted tissue, components such as, for example, inflation balloons can be provided that help to fix the position of the distal treatment end relative to the targeted tissue or which seek to occlude the body lumen to prevent healthy tissue from being exposed to treatment intended for the targeted tissue.

In the event that an inflation balloon is included as part of the catheter, the inflation balloon is generally adhesively attached to the catheter at the appropriate location. Generally, the inflation balloon is positioned over an inflation lumen that extends the length of catheter such that a medical professional can introduce an inflation fluid at the appropriate time. Typically, the inflation balloon is bonded to the catheter following attachment of the catheter tip.

### SUMMARY OF THE DISCLOSURE

The present disclosure relates to improvements in manufacturing catheters that include inflation balloons as part of their particular treatment. Generally, a catheter tip of the present invention is fabricated to include an inflation balloon that is formed/bonded to the catheter tip as part of an initial molding operation. Generally, the catheter tip is formed of a first high durometer polymer while the inflation balloon is formed using a second low durometer polymer. Utilizing a two-step molding operation, the first high durometer polymer can be injected to form the catheter tip and subsequently allowed to cool. Next, the second low durometer polymer is injected to form the inflation balloon and to bond the inflation balloon to the catheter tip so as to create an integrated catheter tip. Once the integrated catheter tip has been formed, the integrated catheter tip can be attached to the catheter body utilizing a conventional attachment method. With the integrated catheter tip now attached to the catheter body, no additional processing steps are required to attach the inflation balloon and consequently, the possibility of additional processing leading to damage to the catheter is removed.

In one aspect, the present invention is directed to a treatment catheter utilizing an inflation balloon as part of a desired treatment procedure. Generally, the treatment catheter comprises an elongated catheter body having a proximal manipulation end and a distal end. An integrated catheter tip is operably joined to the distal end. The integrated catheter tip comprises a molded assembly having a high durometer tip portion and a low durometer inflation balloon. The integrated catheter tip is formed utilizing a two step molding process in which a tip body is formed utilizing a first high durometer polymer that is subsequently allowed to col. The low durometer inflation balloon is formed by an injecting a second low durometer polymer such that the inflation balloon is properly positioned along the integrated catheter tip and no additional joining operations are required.

In another aspect, the present invention is directed to an integrated catheter tip comprising a high durometer tip portion and low durometer inflation balloon. The integrated catheter tip is fabricated using a two-step molding process such that the formation of inflation balloon also results in attachment of the inflation balloon to a high durometer catheter tip. The catheter tip is formed of a first high durometer polymer and subsequently allowed to cool. Once the catheter tip has cooled, a second low durometer polymer is injected to form the inflation balloon while simultaneously bonding the inflation balloon to the catheter tip so as to create an integrated catheter tip. Once the integrated catheter tip has been formed, the integrated catheter tip can be attached to the catheter body utilizing a conventional attachment method.

In yet another aspect, a method for forming a treatment catheter can comprise attaching an integrated catheter tip to a catheter body. Generally, the integrated catheter tip is formed by molding a tip portion from a first high durometer polymer. Next, a second low durometer polymer is injected to form an inflation balloon that is operably coupled to the tip portion.

The above summary of the invention is not intended to describe each illustrated embodiment or every implementation of the present invention. The Figures and the detailed description that follows more particularly exemplify these embodiments.

### BRIEF DESCRIPTION OF THE FIGURES

The invention may be more completely understood in consideration of the following detailed description of various embodiments of the invention in connection with the accompanying drawings, in which:

FIG. 1 is a side view of a treatment catheter according to a representative embodiment of the present disclosure.

FIG. 2 is a side view of an integrated catheter tip according to a representative embodiment of the present disclosure.

FIG. 3 is a section view of the integrated catheter tip of FIG. 2 taken a line 3-3 of FIG. 2.

FIG. 4 is a side view of an integrated catheter tip according to a representative embodiment of the present disclosure.

FIG. 5 is a section view of the integrated catheter tip of FIG. 4 taken a line 5-5 of FIG. 4

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention as defined by the appended claims.

### DETAILED DESCRIPTION OF THE FIGURES

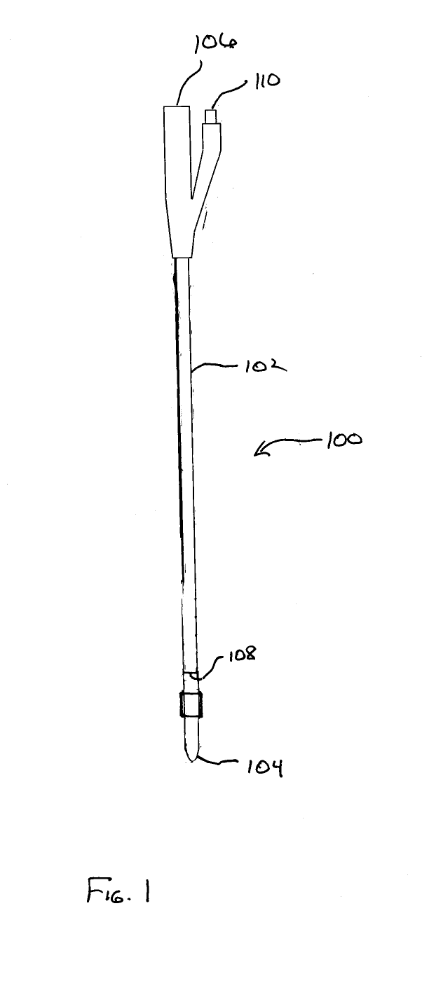

As illustrated in FIG. 1, a treatment catheter 100 of the present invention generally comprises a catheter body 102 and an integrated catheter tip 104. Catheter body 102 generally includes a proximal manipulation end 106 and a distal attachment end 108. Proximal manipulation end typically includes one or more ports 110 in communication with one or more discrete lumens defined within the catheter body. Utilizing ports 110, a medical profession is able to deliver or otherwise administer treatment to a targeted treatment area when the integrated catheter tip 104 is positioned proximate the treatment area.

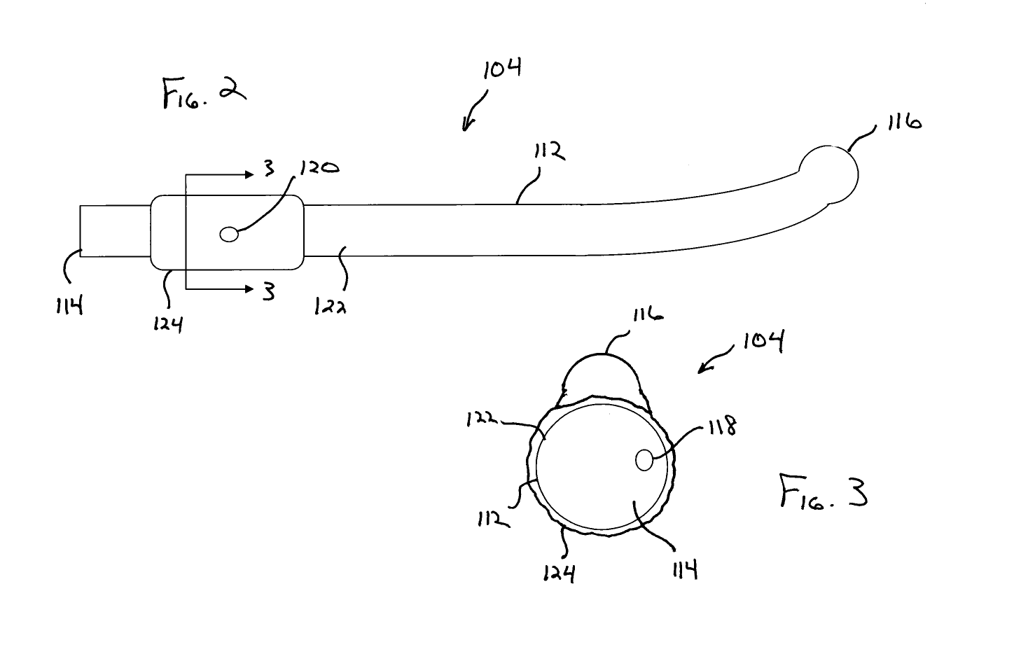

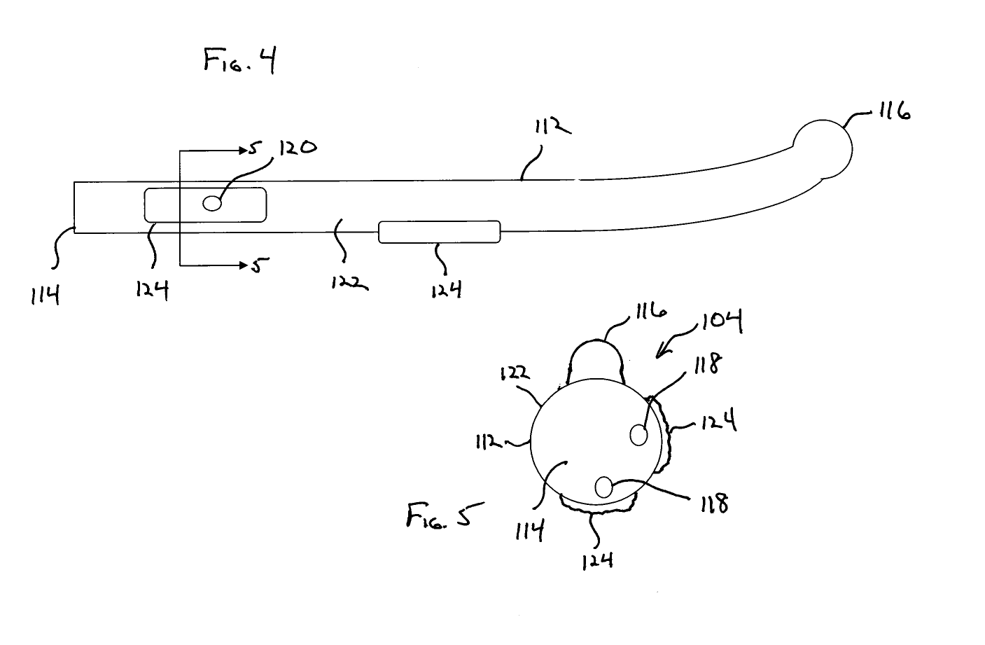

Referring to FIGS. 2 and 3, integrated catheter tip 104 generally comprises a tip body 112 having a tip attachment end 114 and a tip introduction end 116. Tip attachment end 114 generally comprises at least one internal lumen 118 that is fluidly connected to one or more tip apertures 120 on an exterior surface 122 of the tip body 112. Positioned intermediate the tip attachment end 114 and the tip introduction end 116 is an inflation balloon 124. Inflation balloon 124 covers the corresponding tip aperture 120 and sealingly engages the exterior surface 122. In some embodiments, inflation balloon 124 can completely surround the tip body 112 as shown in FIGS. 2 and 3, while in other alternative embodiments, inflation balloon 124 can be located about only a portion of the tip body 112 resulting in an offset configuration as shown in FIGS. 4 and 5. In an alternative embodiment, integrated catheter tip 104 can comprise a plurality of spaced apart inflation balloons 124 as shown in FIGS. 4 and 5.

Generally, the integrated catheter tip 104 is arranged such that tip attachment end 114 is operably connected to the distal attachment end 108 of catheter body 102 to form the treatment catheter 100. With the integrated catheter tip 104 attached to the catheter body 102, a medical professional directs the tip introduction end 116 into a body lumen, whereby the medical professional directs the tip introduction end to a tissue treatment area. With the integrated catheter tip 104 positioned proximate the tissue treatment area, an inflation fluid can be introduced through port 110, through catheter body 102, into the internal lumen 118 and out the tip aperture 120 to inflate the inflation balloon 124.

The integrated catheter tip 104 is generally fabricated utilizing a two-step molding process such that the tip body 112 and inflation balloon 124 comprise a single, integral component. Generally, the tip body 112 is molded using a first high durometer polymer that is allowed to cool within the mold. Next, a second low durometer polymer is injected to form the inflation balloon 124. In forming inflation balloon 124, the inflation balloon 124 is simultaneously attached to the tip body 112 such that integrated catheter tip 104 is formed with no additional bonding or attachment being required.

Although specific examples have been illustrated and described herein, it will be appreciated by those of ordinary skill in the art that any arrangement calculated to achieve the same purpose could be substituted for the specific example shown. This application is intended to cover adaptations or variations of the present subject matter. Therefore, it is intended that the invention be defined by the attached claims and their legal equivalents.

1. An integrated catheter tip, comprising:
a tip body formed of a first high durometer polymer, the tip body defined by an attachment end and an introduction end; and
at least one inflation balloon injection molded to the tip body proximate the attachment end, the inflation balloon formed of a second low durometer polymer,
wherein the tip body and the inflation balloon are sequentially molded using a two-step molding process in which:
the first high durometer polymer is injection molded in a molding operation to form the tip body,
the second low durometer polymer is injection molded in the same molding operation to form at least one inflation balloon molded to the tip body proximate the attachment end.

2. The integrated catheter tip of claim 1, wherein the tip body includes at least one inflation lumen in fluid communication with at least one inflation aperture on an exterior surface of the tip body.

3. The integrated catheter tip of claim 2, wherein the inflation balloon is formed over the inflation lumen and sealingly engages the exterior surface of the tip body.

4. The integrated catheter tip of claim 3, wherein the inflation balloon is positioned to fully surround the exterior surface of the tip body about the inflation aperture.

5. The integrated catheter tip of claim 3, wherein the inflation balloon surrounds only a portion of the exterior surface of the tip body about the inflation aperture.

6. The integrated catheter tip of claim 2, wherein a first inflation balloon is formed over a first inflation lumen and a second inflation balloon is formed over a second inflation aperture, wherein the first and second inflation balloons sealingly engage the exterior surface of the tip body.

7. The integrated catheter tip of claim 6, wherein the first and second inflation balloons are arranged about the exterior surface of the tip body in an offset configuration.

8. A method of forming an integrated catheter tip, comprising:
injection molding a tip body of a first high durometer polymer in a mold, and
injection molding at least one inflation balloon of a second high durometer polymer into the mold containing the tip body such that the inflation balloon is integral to the tip body, wherein the tip body and the inflation balloon are sequentially injection molded as part of the same molding operation.

9. The method of claim 8, wherein the step of molding at least one inflation balloon comprises molding a first inflation balloon and a second inflation balloon such that the first and second inflation balloons are integral to the tip body.

10. The method of claim 9, wherein the step of molding the first inflation balloon and the second inflation balloon comprises positioning the first inflation balloon and the second inflation balloon in an offset configuration.

11. A treatment catheter comprising:
a catheter body having a proximal manipulation end and a distal attachment end; and
an integrated catheter tip including a tip body having a tip attachment end and a tip introduction end, the tip attachment end being operably coupled to the distal attachment end and wherein at least one inflation balloon is injection molded onto the integrated catheter tip so as to integrally mold at least one inflation balloon to the tip body.

12. The treatment catheter of claim 11, wherein the tip body is formed of a first high durometer polymer and the at least one inflation balloon is formed of a second low durometer polymer, wherein the tip body and the inflation balloon are sequentially injection molded using a two-step injection molding process.

13. The treatment catheter of claim 11, wherein the tip body includes at least one inflation lumen in fluid communication with at least one inflation aperture on an exterior surface of the tip body.

14. The treatment catheter of claim 13, wherein the inflation balloon is formed over the inflation lumen and sealingly engages the exterior surface of the tip body.

15. The treatment catheter of claim 14, wherein the inflation balloon is positioned to fully surround the exterior surface of the tip body about the inflation aperture.

16. The treatment catheter of claim 14, wherein the inflation balloon surrounds only a portion of the exterior surface of the tip body about the inflation aperture.

17. The treatment catheter of claim 13, wherein a first inflation balloon is formed over a first inflation lumen and a second inflation balloon is formed over a second inflation aperture, wherein the first and second inflation balloons sealingly engage the exterior surface of the tip body.

18. The treatment catheter of claim 17, wherein the first and second inflation balloons are arranged about the exterior surface of the tip body in an offset configuration.
